(19) United States
(12) Patent Application Publication
(10) Pub. No.: US 2024/0374784 A1
(43) Pub. Date: Nov. 14, 2024

(54) HALLOYSITE-BASED COMPOSITE HEMOSTATIC MATERIAL AND PREPARATION METHOD AND USE THEREOF

(71) Applicant: CHINA UNIVERSITY OF GEOSCIENCES (WUHAN), Wuhan (CN)

(72) Inventors: Huaming YANG, Wuhan (CN); Juan LIAO, Wuhan (CN); Qiying ZHENG, Wuhan (CN)

(73) Assignee: CHINA UNIVERSITY OF GEOSCIENCES (WUHAN), Wuhan (CN)

(21) Appl. No.: 18/391,665

(22) Filed: Dec. 21, 2023

(30) Foreign Application Priority Data
May 10, 2023 (CN) 202310530468.0

(51) Int. Cl. A61L 24/02; A61L 15/18; A61L 15/20; A61L 15/44

(52) U.S. Cl. CPC A61L 24/02; A61L 15/18; A61L 15/20; A61L 15/44; A61L 2300/102; A61L 2300/204; A61L 2300/624; A61L 2400/04

(57) ABSTRACT

In a halloysite-based composite hemostatic material, hydroxylated halloysite is used as a carrier, and the carrier is loaded with aggregation-induced emission (AIE) nanoparticles. The preparation method includes: preparing the hydroxylated halloysite into a hydroxylated halloysite suspension; preparing the AIE nanoparticles into an AIE nanoparticle solution; and thoroughly mixing the hydroxylated halloysite suspension with the AIE nanoparticle solution at a specified temperature and a specified oscillation speed, centrifuging a resulting mixed solution, and lyophilizing a resulting precipitate to obtain an AIE nanoparticles-loaded modified halloysite composite, which is the halloysite-based composite hemostatic material. The surface-hydroxylated halloysite is used to significantly improve a hemostatic effect; and the AIE nanoparticles is functionally loaded at a small amount to further improve hemostatic properties in addition to endowing the halloysite with an antibacterial activity, so as to finally obtain the halloysite-based composite hemostatic material with excellent biocompatibility, high safety, and a prominent antibacterial effect.

### CROSS-REFERENCE TO RELATED APPLICATIONS

This application claims priority to Chinese Patent Application No. 202310530468.0 with a filing date of May 10, 2023. The content of the aforementioned application, including any intervening amendments thereto, is incorporated herein by reference.

### TECHNICAL FIELD

The present disclosure relates to the technical field of hemostatic materials, and in particular to a halloysite-based composite hemostatic material and a preparation method and use thereof.

### BACKGROUND

The development of a hemostatic material with plentiful properties, an excellent hemostatic effect, high biosafety, and a low price has always been a focus of research work. Inorganic hemostatic materials, especially aluminosilicate-based hemostatic agents such as halloysite, zeolite, kaolin, and montmorillonite (MMT), are popular for their advantages such as high efficiency, operability, cost-effectiveness, and minimal tissue responsiveness. Zeolite has an excellent hemostatic effect, but the heat release problem of zeolite during hemostasis has been difficult to overcome.

Over thousands of years, the excellent hemostatic properties and in vivo safety of halloysite nanotubes (HNTs) have been verified in clinical cases. Studies have shown that a procoagulation capacity of HNTs is between a procoagulation capacity of zeolite and a procoagulation capacity of MMT, and is about 2.3 times a procoagulation capacity of kaolin. In addition, researchers have found that a water absorption rate of HNTs is close to a water absorption rate of zeolite (about 80% of a volume of HNTs or zeolite), and is significantly higher than water absorption rates of MMT and kaolin. Therefore, the further functional modification and loading of halloysite to allow synergistic enhancement of a hemostatic effect of halloysite and enrich functions of halloysite are of great significance, especially in the field of wound healing.

### SUMMARY OF PRESENT INVENTION

An objective of the present disclosure is to provide a halloysite-based composite hemostatic material and a preparation method and use thereof in view of the above-mentioned deficiencies of the prior art.

The present disclosure provides a halloysite-based composite hemostatic material, where hydroxylated halloysite is used as a carrier, and the carrier is loaded with aggregation-induced emission (AIE) nanoparticles; and a structural formula of AIE nanoparticles is TTPy as below

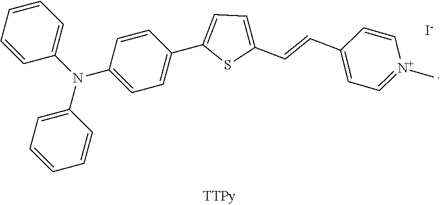 [SMILES: C[n+]1ccc(/C=C/c2ccc(-c3ccc(N(c4ccccc4)c4ccccc4)cc3)s2)cc1 ; [I-]]

In one embodiment, the hydroxylated halloysite is hydroxylated tubular halloysite.

In one embodiment, a preparation method of the hydroxylated halloysite includes:
S1: dispersing halloysite in deionized water, adding sodium hexametaphosphate (SHMP) ((NaPO₃)₆), adjusting a pH of a resulting solution with a NaOH solution, allowing a resulting system to stand, and drying a resulting precipitate to obtain purified halloysite; and
S2: impregnating the purified halloysite with a HCl solution, washing impregnated halloysite with ultrapure water (UPW) until a resulting washing solution is neutral, and drying and grinding washed halloysite to obtain surface-hydroxylated halloysite.

In one embodiment, a concentration of the NaOH solution is 0.1 mol·L⁻¹ to 1 mol·L⁻¹; a mass ratio of the halloysite to the (NaPO₃)₆ is (0.1-20):1; and a concentration of the HCl solution is 0.1 mol·L⁻¹ to 4 mol·L⁻¹.

In one embodiment, the pH is adjusted with the NaOH solution to 11 to 12.

The present disclosure also provides a method for preparing the halloysite-based composite hemostatic material described above, including:
ultrasonically dispersing the surface-hydroxylated halloysite in deionized water to obtain a homogeneous hydroxylated halloysite suspension;
dissolving the AIE nanoparticles in dimethyl sulfoxide (DMSO) to obtain an AIE nanoparticle solution; and
thoroughly mixing the hydroxylated halloysite suspension with the AIE nanoparticle solution at a preset temperature and a preset oscillation speed, centrifuging a resulting mixed solution, and lyophilizing a resulting precipitate to obtain an AIE nanoparticles-loaded modified halloysite composite, which is the halloysite-based composite hemostatic material.

In one embodiment, a mass ratio of the AIE nanoparticles to the surface-hydroxylated halloysite is (0.5-2.0):100.

In one embodiment, a concentration of the AIE nanoparticles is 0.01 mg/mL to 10 mg/mL.

The present disclosure also provides a use of the halloysite-based composite hemostatic material described above, including: preparing a suspension of the halloysite-based composite hemostatic material; impregnating a medical non-woven fabric with the suspension thoroughly stirred, where upper and lower edges of the medical non-woven fabric each are impregnated once; and pressing and oven-drying an impregnated non-woven fabric to obtain a halloysite-loaded hemostatic gauze.

Further, a concentration of the halloysite-based composite hemostatic material in the suspension is 0.1 mg/mL to 100 mg/mL.

In the present disclosure, the surface-hydroxylated halloysite is used to significantly improve a hemostatic effect. The AIE nanoparticles is functionally loaded at a small amount to further improve hemostatic properties in addition to endowing the halloysite with an antibacterial activity, so as to finally obtain the halloysite-based composite hemostatic material with excellent biocompatibility, high safety, and a prominent antibacterial effect. Moreover, the preparation method of the present disclosure involves simple steps and easy operations, and is conducive to large-scale production.

In the present disclosure, the medical non-woven fabric is combined with the halloysite-based composite hemostatic material through impregnation to prepare a medical hemostatic gauze product with excellent hemostatic properties, prominent biocompatibility, high safety, and antibacterial activity, which involves a simple preparation method and is conducive to industrial production.

### BRIEF DESCRIPTION OF THE DRAWINGS

FIG. 1 shows a structure of AIE nanoparticles;

FIG. 2 shows morphologic images of Raw HNTs, Example 1, and Example 2;

FIG. 3 shows Fourier Transform Infrared (FTIR) spectra of Raw HNTs, Example 1, and Example 2;

FIG. 4 shows pictures of the blank gauze and the gauze products prepared in Examples 3 to 5 of the present disclosure;

FIG. 5 shows morphologic images of surfaces of the blank gauze and the halloysite-based composite-loaded hemostatic gauzes prepared in Examples 3 to 5 of the present disclosure and a Quikclot gauze (including kaolinite as a main component);

FIG. 6 shows morphologic images of cross sections of the blank gauze and the halloysite-based composite-loaded hemostatic gauzes prepared in Examples 3 to 5 of the present disclosure and a Quikclot gauze;

FIG. 7a and FIG. 7b show antibacterial results of the blank gauze and the halloysite-based composite-loaded hemostatic gauzes prepared in Examples 3 to 5 of the present disclosure;

FIG. 8 shows cytotoxicity results of the blank gauze and the halloysite-based composite-loaded hemostatic gauzes prepared in Examples 3 to 5 of the present disclosure;

FIG. 9 shows bleeding times in the mouse liver injury model treated by the blank gauze and the halloysite-based composite-loaded hemostatic gauzes prepared in Examples 3 to 5 of the present disclosure and a Quikclot gauze; and

FIG. 10 shows blood losses in the mouse liver injury model treated with the blank gauze and the halloysite-based composite-loaded hemostatic gauzes prepared in Examples 3 to 5 of the present disclosure and a Quikclot gauze.

### DETAILED DESCRIPTION OF THE EMBODIMENTS

The technical solutions of the present disclosure are described in further detail below with reference to the specific embodiments and accompanying drawings, but the present disclosure is not limited thereto.

The halloysite used in the examples of this specification is a halloysite nanoclay product of Sigma-Aldrich of the United States, and the halloysite nanoclay product has a molecular formula of Al₂Si₂O₅(OH)₄·2H₂O, a molecular weight of 294.19, and a density of 2.53 g/cm³.

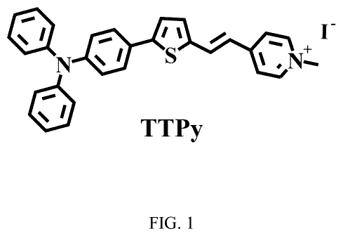

The structure formula of the AIE nanoparticles used in the examples is TTPy, which is prepared by Guangzhou Tanshui Technology Co., Ltd according to the structural formula designed by the applicants as shown in FIG. 1.

### Preparation of Halloysite

Preparation of a halloysite raw material: Halloysite was thoroughly ground to obtain the halloysite raw material denoted as Raw HNTs.

### Preparation of an AIE Nanoparticle Suspension

10 mg of AIE nanoparticles were dissolved in 1 mL of DMSO to obtain a 10 mg/mL AIE nanoparticle solution.

### Preparation of Modified Halloysite

### Example 1

Halloysite modification: 10 g of the halloysite raw material (Raw HNTs) was taken and added to 1,000 mL of deionized water to obtain a first mixture. The first mixture was magnetically stirred for about 1 h and then ultrasonically treated for about 1 h. This process was repeated until the Raw HNTs were fully dispersed. Then 0.5 g of SHMP ((NaPO₃)₆) was added to obtain a second mixture, and the second mixture was vigorously stirred for about 1 h to make (NaPO₃)₆ fully dispersed to obtain a first system. Then a pH of the first system was adjusted with a 1 mol L⁻¹ NaOH solution to about 11 to obtain a second system. The second system was allowed to stand for about 3 h and then subjected to suction filtration to obtain a solid precipitate, and the solid precipitate was dried at 60° C. for 24 h to obtain purified halloysite. The purified halloysite was impregnated with a 4 mol·L⁻¹ HCl solution at room temperature for 3 h, then washed several times with UPW until a resulting washing solution was neutral, and finally dried and ground to obtain the modified halloysite, i.e. HNTs.

### Preparation of an AIE Nanoparticles-Loaded Modified Halloysite Composite

### Example 2

693 μL of deionized water was added to 267 μL of a 100 mg/mL HNTs suspension, and then 40 μL of a 10 mg/mL of AIE nanoparticle solution was added to obtain 1 mL of a mixed solution. The mixed solution was thoroughly mixed at 37° C. and 1,000 rpm for 3 h in a thermomixer, and then centrifuged at 14,000 rpm for 10 min, and a resulting precipitate was lyophilized for 24 h to obtain the AIE nanoparticles-loaded modified halloysite composite, which was denoted as HNTs-TTPy.

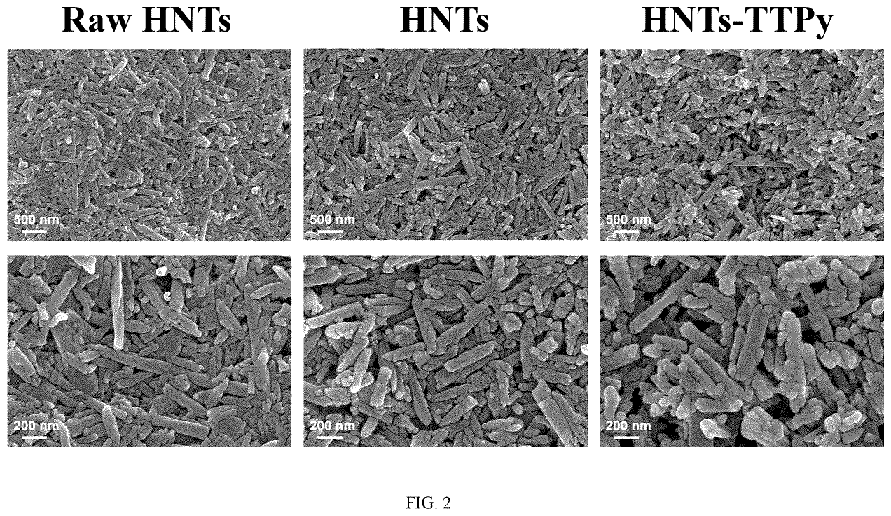

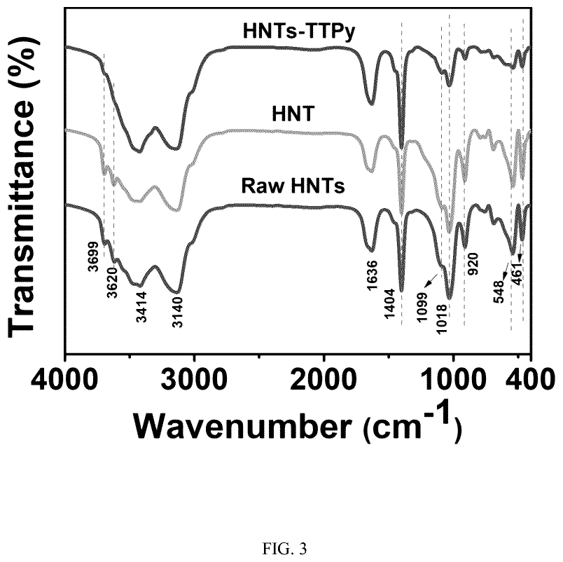

FIG. 2 shows morphologic images of Raw HNTs, Example 1, and Example 2. It can be seen from FIG. 2 that Examples 1 and 2 have smaller sizes and larger exposed surfaces than Raw HNTs. FIG. 3 shows Fourier Transform Infrared (FTIR) spectra of Raw HNTs, Example 1, and Example 2. It can be seen from FIG. 3 that, compared with Raw HNTs, stretching vibration peaks (1,099 cm⁻¹ and 1,018 cm⁻¹) of Si—O, a bending vibration peak (920 cm⁻¹) of Al—OH, and stretching vibration peaks of Si—O and Al—O (548 cm⁻¹ and 461 cm⁻¹) in Example 1 are significantly enhanced, indicating that hydroxyl groups on a surface of the halloysite are increased. Characteristic peaks of Example 3 are lower than characteristic peaks of Example 2, which may be caused by the bonding of the AIE nanoparticles to a surface of the halloysite.

### Preparation of Halloysite-Loaded Hemostatic Gauzes

### Example 3

3 mL of a 100 mg/mL Raw HNTs suspension was placed in a beaker, 27 mL of deionized water was added, and a resulting system was thoroughly stirred at 500 rpm for 1 h to obtain a homogeneous suspension. A piece of a non-woven gauze (area: 10*9.5 cm²) was cut and directly impregnated with the homogeneous suspension to make a Raw HNTs powder adhered to a surface of the non-woven gauze, where upper and lower edges of the non-woven gauze each were impregnated once with a total impregnation time of about 2 s. An impregnated non-woven gauze was pressed by a roller press with a distance of 0.05 mm between upper and lower rollers of the roller press to enhance the adhesion between the Raw HNTs powder and the non-woven gauze. Finally, a pressed non-woven gauze was hung by dovetail clips in an oven at 60° C. and blow-dried to obtain a gauze with halloysite composite, which was denoted as Raw HNTs Gauze.

### Example 4

3 mL of a 100 mg/mL HNTs suspension was placed in a beaker, 27 mL of deionized water was added, and a resulting system was thoroughly stirred at 500 rpm for 1 h to obtain a homogeneous suspension. A piece of a non-woven gauze (area: 10*9.5 cm²) was cut and directly impregnated with the homogeneous suspension to make an HNTs powder adhered to a surface of the non-woven gauze, where upper and lower edges of the non-woven gauze each were impregnated once with a total impregnation time of about 2 s. An impregnated non-woven gauze was pressed by a roller press with a distance of 0.05 mm between upper and lower rollers of the roller press to enhance the adhesion between the HNT powder and the non-woven gauze. Finally, a pressed non-woven gauze was hung by dovetail clips in an oven at 60° C. and blow-dried to obtain a gauze with modified halloysite composite, which was denoted as HNTs Gauze.

### Example 5

3 mL of a 100 mg/mL HNT suspension was placed in a beaker, 26.55 mL of deionized water was added, a 10 mg/mL AIE nanoparticle solution was added under stirring at 500 rpm, and a resulting system was further stirred thoroughly for 1 h to obtain a homogeneous suspension. A piece of a non-woven gauze (area: 10*9.5 cm²) was cut and directly impregnated with the homogeneous suspension to make a composite powder adhered to a surface of the non-woven gauze, where upper and lower edges of the non-woven gauze each were impregnated once with a total impregnation time of about 2 s. An impregnated non-woven gauze was pressed by a roller press with a distance of 0.05 mm between upper and lower rollers of the roller press to enhance the adhesion between the composite powder and the non-woven gauze. Finally, a pressed non-woven gauze was hung by dovetail clips in an oven at 60° C. and blow-dried to obtain a gauze with an AIE nanoparticles-loaded modified halloysite composite, which was denoted as HNTs-TTPy Gauze.

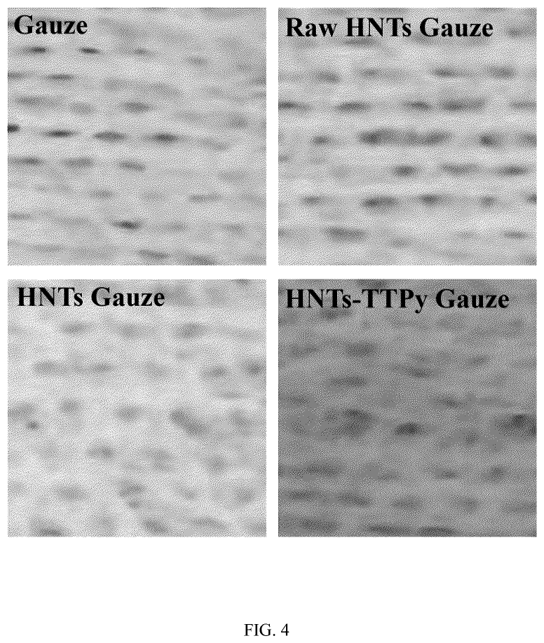

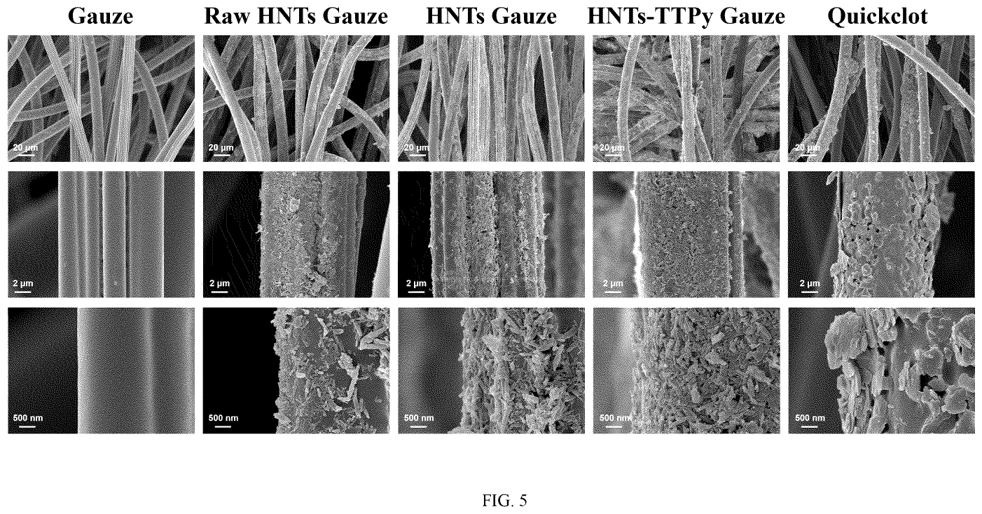

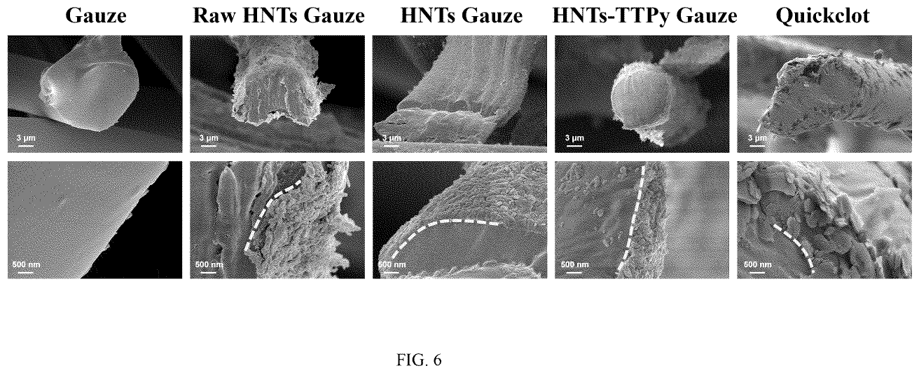

FIG. 4 shows pictures of the gauze products obtained by impregnating a gauze with a suspension of the halloysite-based composite hemostatic material, and it can been from this figure that a color of the HNTs-TTPy Gauze is obviously darkened, indicating that the halloysite-based composite hemostatic material is successfully loaded on the gauze. It can be seen from morphologic images of surfaces and cross sections of the hemostatic gauzes prepared in Examples 3 to 5 in FIG. 5 and FIG. 6 that the tubular halloysite-based composite hemostatic material is successfully loaded on the gauze; and flaky kaolinite is loaded in Quickclot.

Loads of the hemostatic materials in the gauze products obtained in Examples 3 to 5 were shown in Table 1.

TABLE 1
Loads of hemostatic materials in the gauze products

| | Mass before | Mass after | Load | Loading |
|---|---|---|---|---|
| Sample | loading (g) | loading (g) | (g) | density (g/m²) |
| Example 3 | 0.5119 | 0.5330 | 0.0211 | 2.2211 |
| Example 4 | 0.4951 | 0.5139 | 0.0188 | 1.9789 |
| Example 5 | 0.5055 | 0.5608 | 0.0553 | 5.8211 |

### In Vitro Antibacterial Experiment:

Staphylococcus aureus (S. aureus) (ATCC 25923) was adopted as a gram-positive bacterial model to evaluate an antibacterial activity of a sample. A single S. aureus strain was dispersed in 5 mL of a Luria-Bertani liquid medium and shaken at 37° C. for 8 h to obtain a bacterial suspension with an initial bacterial concentration of 2× 106 CFU/mL. The bacterial suspension was serially diluted 103-fold with phosphate buffered saline (PBS). Then 1 mL of a diluted bacterial suspension was mixed with 20 mg of a gauze cut into pieces, and a resulting bacterial sample was incubated in the dark for 5 min and then irradiated under white light at 100 mw/cm² for 30 min. The bacterial sample was then serially diluted 10-fold with PBS, and 100 μL of a diluted bacterial sample was spread on a corresponding solid agar plate and then incubated at 37° C. for 14 h to 16 h. A bacterial survival rate was used as an index to evaluate an antibacterial activity of a material against bacteria.

$$Bacterial survival rate = average number of colonies in a sample / average number of colonies in a control group \times 100 \%$$

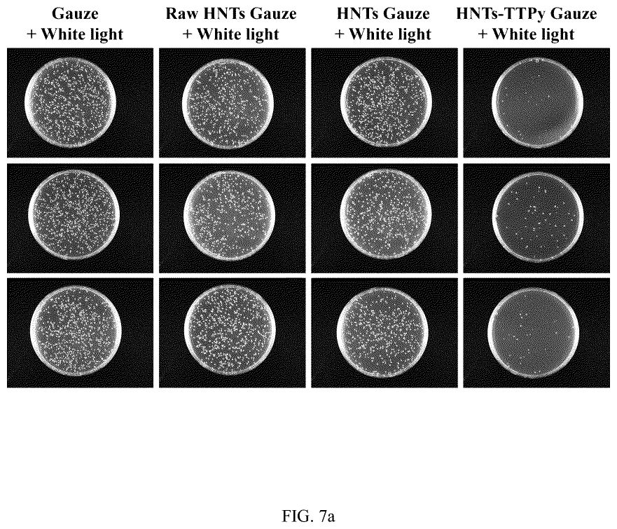

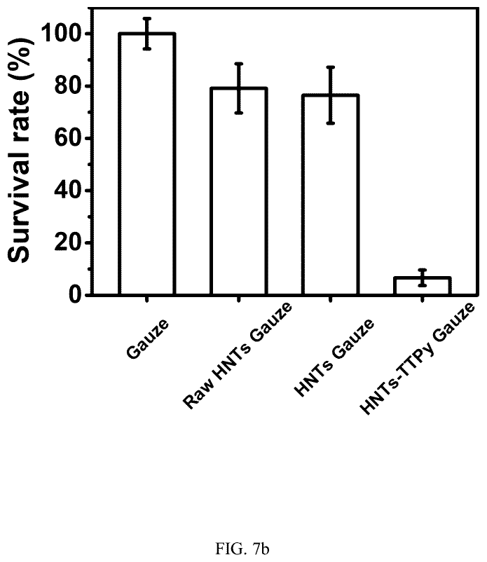

Antibacterial effects of a blank gauze and Examples 3 to 5 against S. aureus were shown in FIG. 7a and FIG. 7b. It can be seen from the results of bacterial coating and bacterial counting in FIG. 7a and FIG. 7b that Raw HNTs Gauze and HNTs Gauze exhibit weak antibacterial effects against S. aureus compared with the blank gauze, which may be related to the contact and adhesion of clay minerals on a surface of bacteria. Due to the isolation of bacteria from an outside environment, the normal metabolism of the bacteria is affected, which further leads to death of the bacteria. HNTs-TTPy Gauze has a significant antibacterial effect after light irradiation, indicating that, after being compounded with TTPy, the clay mineral halloysite can produce a large amount of reactive oxygen species (ROS) under light irradiation to effectively kill bacteria.

### Cytotoxicity Experiment:

In this experiment, a Cell Counting Kit-8 (CCK-8) method was used to analyze the cytotoxicity of a sample with human immortalized fibroblasts (BJ cells) as a research object. A complete medium for cultivating the BJ cells was prepared with an RPMI-1640 basal medium, 1% of penicillin-streptomycin, and 10% of fetal bovine serum (FBS). A first BJ cell suspension normally cryopreserved was constantly shaken in a 37° C. water bath until the first BJ cell suspension was completely thawed, 1 mL of the first BJ cell suspension was taken and added to a 15 mL centrifuge tube filled with 10 mL of the complete medium, and the centrifuge tube was thoroughly shaken and then centrifuged at 1,000 rpm for 5 min. A resulting supernatant was removed, a resulting cell pellet was resuspended with the complete medium to obtain a second cell suspension, and the second cell suspension was transferred to a Petri dish. Cells were cultivated to an excellent adherent growth state, then trypsin was added for digestion to make the cells detached, and the cells were resuspended to obtain a third cell suspension. a density of the third cell suspension was adjusted to obtain a fourth cell suspension, and then the fourth cell suspension was inoculated into a 96-well plate at a density of 1×10⁴ cells/well and cultivated in 37° C. and 5% CO2 incubator until cells were in an excellent adherent growth state. The original medium was discarded, 100 μL of a fresh complete medium was added to a control well, and 100 μL of a sample solution was added at different concentrations to each test well. The cells were further cultivated for 24 h, and then the cytotoxicity was determined as follows: a 10% CCK-8 reagent-containing complete medium was added to each well, cells were further cultivated for 1 h, and an absorbance (OD) value at 450 nm was determined by a microplate reader. A well without a sample was set as a blank control group. A cell survival rate was calculated by determining the absorbance. The cell survival rate was calculated as follows:

$$Cell survival rate = ( {OD}_{test well} - {OD}_{blank well} ) / ( {OD}_{control well} - {OD}_{blank well} ) \times 100 \% .$$

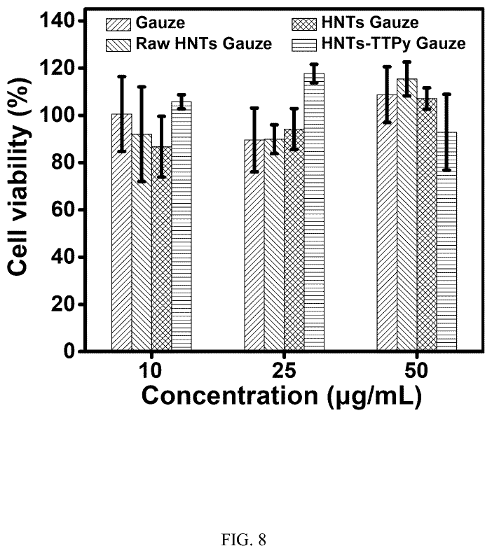

The concentrations of 10 μg/mL, 25 μg/mL, and 50 μg/mL were set to evaluate the cytotoxicities of the blank gauze and Examples 3 to 5, and test results of the cytotoxicity experiment were shown in FIG. 8. Among cytotoxicity levels, when a cell survival rate is greater than 75%, it is determined that a biosafety level 1 is reached. Cell survival rates of the blank gauze and Examples 3 to 5 in the experimental concentration range all are higher than 75%, indicating that both HNTs Gauze and HNTs-TTPy Gauze have excellent biocompatibility.

### In Vivo Liver Hemostasis Experiment:

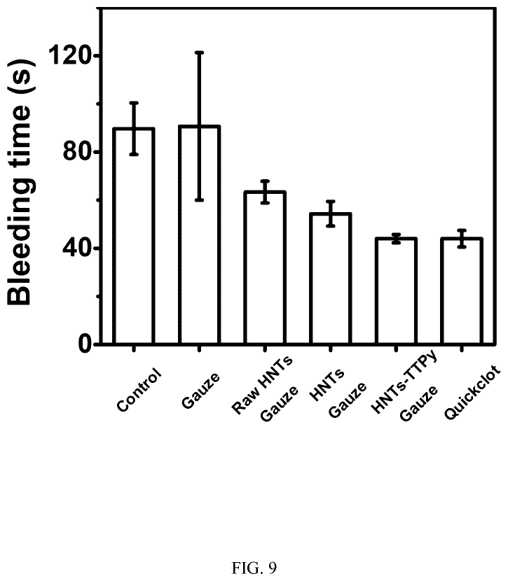

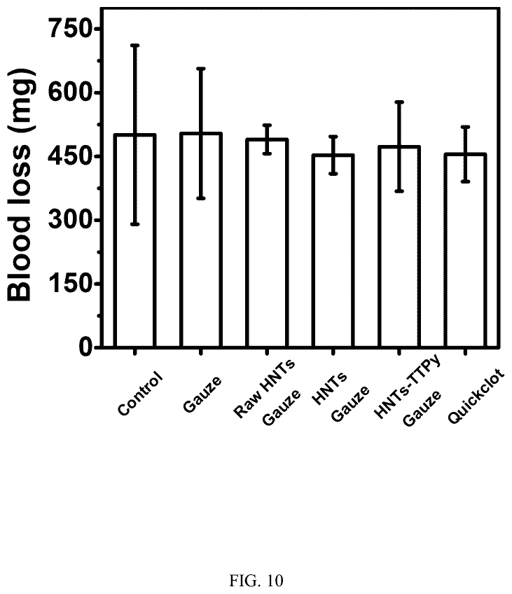

8-10 week-old male Kunming mice were selected and randomly grouped according to body weights, with 5 mice in each group. Each mouse was anesthetized and fixed, an abdominal cavity of the mouse was opened, and a wound of about 1 cm was cut with a scalpel on a left lobe tissue of a liver. A bleeding left lobe of the liver was covered with a gauze sample. A bleeding time was recorded, and a mass change of the gauze sample was measured to calculate a blood loss. A bleeding time and a blood loss of each sample were shown in Table 2, FIG. 9, and FIG. 10.

TABLE 2
Bleeding times and blood losses of hemostatic gauzes

| | | Bleeding | Blood |
|---|---|---|---|
| Sample | Material | time (s) | loss (mg) |
| Control group | No material is added | 89.67 ± | 500.90 ± |
| | | 10.69 | 210.30 |
| Blank gauze | Additive-free gauze | 90.67 ± | 504.09 ± |
| | | 30.66 | 152.70 |
| Example 3 | Raw HNTs-containing | 63.33 ± | 489.96 ± |
| | gauze | 4.51 | 33.31 |
| Example 4 | HNTs-containing gauze | 54.33 ± | 453.12 ± |
| | | 5.13 | 43.69 |
| Example 5 | HNTs-TTPy-containing | 44.00 ± | 472.90 ± |
| | gauze | 1.73 | 104.95 |
| Quikclot | Quikclot gauze | 44.00 ± | 455.00 ± |
| | | 3.46 | 64.29 |

It can be seen from Table 2, FIG. 9, and FIG. 10 that HNTs Gauze in Example 4 has a shortened bleeding time compared with Example 3 (Raw HNTs Gauze), indicating that the surface hydroxylation of halloysite may increase hydroxyl groups exposed on a surface of the halloysite to improve the hemostatic properties. HNTs-TTPy Gauze in Example 5 exhibits further improved hemostatic properties compared with Examples 3 and 4. It indicates that the increased hydroxyl groups exposed on the surface of the halloysite can increase a load of the AIE nanoparticles in addition to improving the hemostatic properties, and the positively-charged AIE nanoparticles can reduce the hemolysis of red blood cells (RBCs) by neutralizing negative charges on a surface of some HNTs, which can improve the biocompatibility and hemostatic properties of HNTs-TTPy Gauze. Therefore, the surface-hydroxylated halloysite and TTPy in HNTs-TTPy Gauze can allow the synergistic improvement of hemostatic properties. In addition, a hemostasis rate of HNTs-TTPy Gauze is comparable to a hemostasis rate of the existing hemostatic gauze (Quikclot) on the market, and compared with the control group, HNTs-TTPy Gauze has a significantly-improved hemostasis rate, a reduced blood loss, and an excellent antibacterial activity.

What is not mentioned above can be acquired in the prior art.

Although some specific embodiments of the present disclosure have been described in detail by way of examples, those skilled in the art will appreciate that the above examples are provided for illustration only and not for limiting the scope of the present disclosure. A person skilled in the art can make various modifications or supplements to the specific embodiments described or replace them in a similar manner, but it may not depart from the direction of the present disclosure or the scope defined by the appended claims. Those skilled in the art should understand that any modification, equivalent replacement, and improvement that are made to the above embodiments according to the technical essence of the present disclosure shall be included in the protection scope of the present disclosure.

What is claimed is:

1. A halloysite-based composite hemostatic material, wherein hydroxylated halloysite is used as a carrier, and the carrier is loaded with aggregation-induced emission (AIE) nanoparticles; and a structural formula of the AIE nanoparticles is TTPy as below

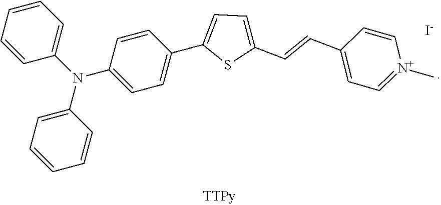 [SMILES: C[n+]1ccc(/C=C/c2ccc(-c3ccc(N(c4ccccc4)c4ccccc4)cc3)s2)cc1 ; [I-]]

2. The halloysite-based composite hemostatic material according to claim 1, wherein the hydroxylated halloysite is hydroxylated tubular halloysite.

3. The halloysite-based composite hemostatic material according to claim 1, wherein a preparation method of the hydroxylated halloysite comprises:
S1: dispersing halloysite in deionized water, adding sodium hexametaphosphate (SHMP) ((NaPO₃)₆), adjusting a pH of a resulting solution with a NaOH solution, allowing a resulting system to stand, and drying a resulting precipitate to obtain purified halloysite; and
S2: impregnating the purified halloysite with a HCl solution, washing impregnated halloysite with ultrapure water (UPW) until a resulting washing solution is neutral, and drying and grinding washed halloysite to obtain surface-hydroxylated halloysite.

4. The halloysite-based composite hemostatic material according to claim 3, wherein a concentration of the NaOH solution is 0.1 mol·L⁻¹ to 1 mol·L⁻¹; a mass ratio of the halloysite to the (NaPO₃)₆ is (0.1-20):1; and a concentration of the HCl solution is 0.1 mol·L⁻¹ to 4 mol·L⁻¹.

5. The halloysite-based composite hemostatic material according to claim 3, wherein the pH is adjusted with the NaOH solution to 11 to 12.

6. A method for preparing the halloysite-based composite hemostatic material according to claim 1, comprising:
ultrasonically dispersing the surface-hydroxylated halloysite in deionized water to obtain a homogeneous hydroxylated halloysite suspension;
dissolving the AIE nanoparticles in dimethyl sulfoxide (DMSO) to obtain an AIE nanoparticle solution; and
thoroughly mixing the hydroxylated halloysite suspension with the AIE nanoparticle solution at a preset temperature and a preset oscillation speed, centrifuging a resulting mixed solution, and lyophilizing a resulting precipitate to obtain an AIE nanoparticles-loaded modified halloysite composite, which is the halloysite-based composite hemostatic material.

7. The method according to claim 6, wherein a mass ratio of the AIE nanoparticles to the surface-hydroxylated halloysite is (0.5-2.0):100.

8. The method according to claim 6, wherein a concentration of the AIE nanoparticles is 0.01 mg/mL to 10 mg/mL.

9. A use of the halloysite-based composite hemostatic material according to claim 1, comprising:
preparing a suspension of the halloysite-based composite hemostatic material;
impregnating a medical non-woven fabric with the suspension thoroughly stirred, wherein upper and lower edges of the medical non-woven fabric each are impregnated once; and
pressing and oven-drying the impregnated non-woven fabric to obtain a halloysite-loaded hemostatic gauze.

10. The use according to claim 9, wherein a concentration of the halloysite-based composite hemostatic material in the suspension is 0.1 mg/mL to 100 mg/mL.
